(19) United States
(12) Patent Application Publication
(10) Pub. No.: US 2008/0317831 A1
(43) Pub. Date: Dec. 25, 2008

(54) HEMOSTATIC SPONGE AND METHOD OF MAKING THE SAME

(76) Inventor: Denny Lo, Bethlehem, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 12/140,356

(22) Filed: Jun. 17, 2008

Related U.S. Application Data
(60) Provisional application No. 60/936,768, filed on Jun. 21, 2007.

(51) Int. Cl. A61K 9/70

(52) U.S. Cl. 424/447

(57) ABSTRACT

A device for promoting the clotting of blood includes a web of non-woven fibers of a polymer having a hemostatic agent disposed on the fibers. The fibers are randomly arranged to form the web. When the device is applied to a bleeding wound, at least a portion of the hemostatic agent comes into contact with blood to cause the blood to clot. A hemostatic sponge includes a melt-blown non-woven fibrous web of polymer material and a hemostatic agent that is attached to the fibers. A method of making a hemostatic sponge includes the steps of melting a polymer and combining the polymer with a hot air stream. A hemostatic agent is added to the melt. The melt with the hemostatic agent is then drawn into fibers and collected as a web.

### CROSS-REFERENCE TO RELATED APPLICATION

This application claims the benefit of U.S. Provisional Patent Application No. 60/936,768, filed on Jun. 21, 2007, the contents of which are incorporated herein by reference in their entirety.

### TECHNICAL FIELD

The present invention is generally directed to hemostatic sponges and methods of making hemostatic sponges and, more particularly, to hemostatic sponges made using melt blown technology.

### BACKGROUND OF THE INVENTION

Blood is a liquid tissue that includes red cells, white cells, corpuscles, and platelets dispersed in a liquid phase. The liquid phase is plasma, which includes acids, lipids, solublized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, wounds can be inflicted as the result of trauma. Often bleeding is associated with such wounds. In some circumstances, the wound and the bleeding are minor, and normal blood clotting functions in addition to the application of simple first aid are all that is required. First aid may include applying pressure to the wound with a sponge or similar device to facilitate clotting functions. Unfortunately, however, in other circumstances substantial bleeding can occur. While sponges may still be utilized, these situations usually require specialized equipment and additional materials as well as personnel trained to administer appropriate aid.

Bleeding can also be a problem when the trauma is the result of a surgical procedure. Apart from suturing or stapling an incision or internally bleeding area, bleeding encountered during surgery is often controlled using sponges or other materials used to exert pressure against the bleed site and/or absorb the blood. However, when the bleeding becomes excessive, these measures may not be sufficient to stop the blood flow.

In treating any type of bleeding wound, the sponges employed generally include a substrate and a hemostatic agent in powder or particulate form on the substrate. In some sponges, the powder or particulate is held onto the substrate (typically pre-manufactured) with a binder material such as glycerin. With regard to these types of sponges, the hemostatic agents are sometimes released from the substrates when the sponges are wetted with blood or other body fluids. If the agents are water soluble, as glycerin is, then the agents may be released into the blood stream during treatment of the wounded person.

In other sponges, the powder or particulate can be held onto the pre-manufactured substrate using mechanical means such as trapping the powder or particulate in a fiber matrix of the substrate. The fiber matrix may resemble a mesh. In still other sponges, the substrate is paper or other cellulose-based material, and the powder or particulate is embedded into this material. Particularly when the hemostatic agent is trapped in a substrate that is a fiber matrix or mesh, flexing of the substrate may cause the fibers or strands of the mesh to move relative to each other, thereby releasing the hemostatic agent into the wound.

In any sponge, if the hemostatic agent is adhered in such a way that it is prevented from making direct contact with blood, then the hemostatic properties of the sponge are diminished. In particular, unless the hemostatic agent is secured to the material of the substrate, the sponge is not utilized to its fullest potential and hemostatic agent is wasted.

### SUMMARY OF THE INVENTION

In one aspect, the present invention resides in a device for promoting the clotting of blood. This device comprises a web of non-woven fibers of a polymer having a hemostatic agent disposed on the fibers. As used herein, the term “web” is intended to mean a continuous sheet of material that is manufactured or being manufactured using the techniques and apparatus described below. The fibers are randomly arranged to form the web. When the device is applied to a bleeding wound, at least a portion of the hemostatic agent comes into contact with blood to cause the blood to clot.

In another aspect, the present invention resides in a hemostatic sponge that comprises a melt-blown non-woven fibrous web of polymer material and a hemostatic agent attached to the fibers. When this hemostatic sponge is used to treat a bleeding wound, at least a portion of the hemostatic agent comes into contact with blood to cause the blood to clot.

In another aspect, the present invention resides in a method of making a hemostatic sponge. In making such a sponge, a polymer is melted and combined with a hot air stream. A hemostatic agent is added to the melt (either by being added to the hot air stream or by being added in a separate stream). The melt with the hemostatic agent is then drawn into fibers and collected as a web.

One advantage of the present invention is that the retention of the hemostatic agent on the substrate is improved in comparison with sponges of the prior art. In particular, because the hemostatic agent is effectively “melted” into the material of the fibrous web, the hemostatic agent is securely held on the sponge, which thereby eliminates or at least decreases the possibility of the hemostatic agent being dislodged from the fibrous web and being deposited into the wound. Thus, there is no need for irrigation of the wound to remove any loose hemostatic agent.

Another advantage of the present invention is that because the hemostatic agent is securely held on the fibers of the web without a binder, there is no contamination of the blood with the binder. While most sponges that utilize binders to hold the hemostatic agent to the substrate are generally recognized as safe according to medical standards, not having a binder means that no foreign materials are introduced into the blood stream.

### BRIEF DESCRIPTION OF THE DRAWINGS

FIG. 1 is a perspective view of a hemostatic sponge of the present invention.

FIG. 2 is a fiber of the hemostatic sponge of FIG. 1, the fiber containing a particle of hemostatic agent.

FIG. 3 is a flowchart representation of a melt blowing process, of the present invention, for fabricating the hemostatic sponge of the present invention.

FIG. 4 is a side sectional view of a screw extruder that can be used for the melt blowing process of the present invention.

FIG. 5 is a schematic representation of a metering pump that can be used for the melt blowing process of the present invention.

FIG. 6 is a schematic representation of the collection of formed fibers having hemostatic agent incorporated therein for use in the hemostatic sponge of the present invention.

FIG. 7 is a perspective view of formed fibers having hemostatic agent incorporated therein being rolled between calender and anvil rolls and collected as a web.

FIG. 8 is a schematic representation of a nosepiece of a die assembly of the present invention.

FIG. 9 is one embodiment of a nosepiece of the present invention.

FIG. 10 is another embodiment of a nosepiece of the present invention.

### DETAILED DESCRIPTION OF THE PREFERRED EMBODIMENTS

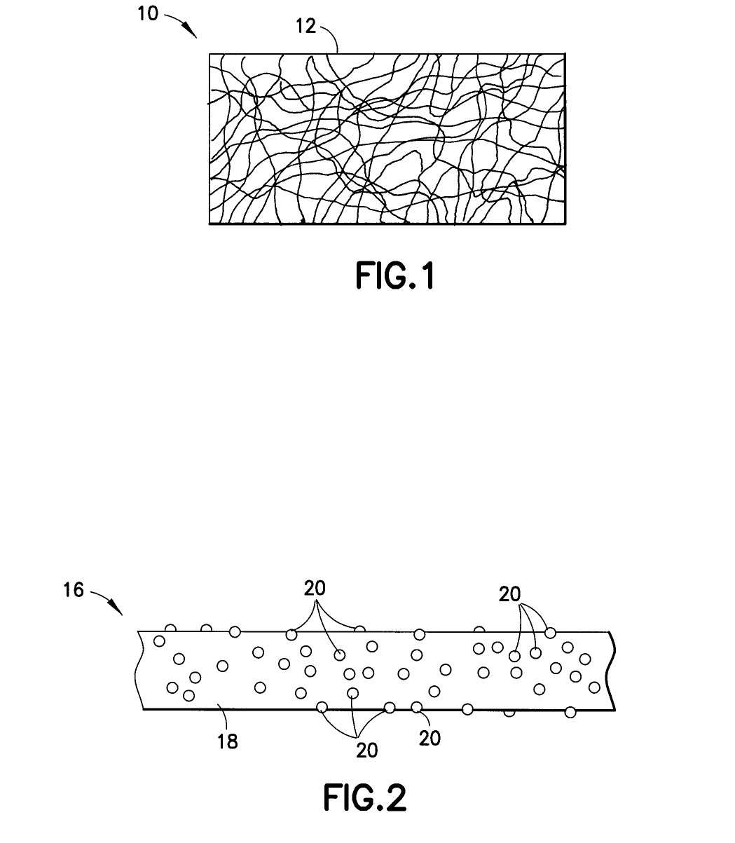

As shown in FIG. 1, a hemostatic sponge made using a melt blowing technique is shown generally at 10 and is hereinafter referred to as “sponge 10.” The sponge 10 includes a substrate 12 of non-woven fibers. A hemostatic agent is disposed on the fibers. The hemostatic agent may be bonded thereto, incorporated therein, impregnated therein, or otherwise held fast on the fibers. Preferably, the hemostatic agent is embedded into or at least coated onto the fibers. Due to the use of the melt blowing technique, the fibers are self-bonded into a non-woven web.

Referring to FIG. 2, a fiber of the substrate is shown at 16. The fiber 16 is a strand of material 18 containing particles 20 of the hemostatic agent. The material 18 may be a polymer selected from the group consisting of thermoplastics, elastomers, other types of polymers, combinations of the foregoing, or any other suitable material. Because of the nature of the melt blowing technique, the formed fibers 16 are non-woven and generally randomly arranged to form the web. Because of the generally random formation of the web, the fibers 16 are linked with each other at various places along the length of each fiber. The fiber 16 is about 0.05 mm (millimeters) to about 0.8 mm in diameter and preferably about 0.2 mm to about 0.5 mm in diameter.

Various types of polymers may be used depending upon the desired characteristics of the finished sponge. Exemplary polymers that may be used to form the fibers 16 include polypropylenes, polyesters, and combinations of the foregoing. Other polymers that may be used in forming the fibers 16 of the present invention include, but are not limited to, acrylonitrile butadiene styrene, polyamides, polylactic acid, polyacrylates, and the like, combinations of the foregoing, and combinations of the foregoing with polypropylenes and/or polyesters.

Any suitable hemostatic agent may be used to form the particles 20. Materials that may be used as hemostatic agents include clays or other silica-based materials that, when brought into contact with a bleeding wound, can minimize or stop blood flow, thereby facilitating clotting. The present invention is not limited to clay, however, as other materials such as bioactive glasses, zeolite, biological hemostats, chitin, chitosan, molecular sieve materials, diatomaceous earth, combinations of the foregoing, and the like are within the scope of the present invention and can be used in conjunction with the clay or separately as the hemostatic agent.

In one embodiment of the present invention, the clay is kaolin, which includes the mineral “kaolinite.” Although the term “kaolin” is used hereinafter to describe the present invention, it should be understood that kaolinite may also be used in conjunction with or in place of kaolin. The present invention is also not limited with regard to kaolin or kaolinite, however, as other materials are within the scope of the present invention. Such materials include, but are not limited to, attapulgite, bentonite, combinations of the foregoing, combinations of the foregoing with kaolin, and the like.

The clay may be Edgar's plastic kaolin (hereinafter “EPK”), which is a water-washed kaolin clay that is mined and processed in and near Edgar, Fla. Edgar's plastic kaolin has desirable plasticity characteristics, is castable, and when mixed with water produces a thixotropic slurry.

The kaolin material of the present invention may be mixed with or otherwise used in conjunction with other materials to provide additional clotting functions and/or improved efficacy. Such materials include, but are not limited to, magnesium sulfate, sodium metaphosphate, calcium chloride, dextrin, combinations of the foregoing materials, and hydrates of the foregoing materials.

Various materials may be mixed with, associated with, or incorporated into the kaolin to maintain an antiseptic environment at the wound site or to provide functions that are supplemental to the clotting functions of the clay. Exemplary materials that can be used include, but are not limited to, pharmaceutically-active compositions such as antibiotics, antifungal agents, antimicrobial agents, anti-inflammatory agents, analgesics, antihistamines (e.g., cimetidine, chloropheniramine maleate, diphenhydramine hydrochloride, and promethazine hydrochloride), compounds containing silver ions, compounds containing copper ions, combinations of the foregoing, and the like. Other materials that can be incorporated to provide additional hemostatic functions include ascorbic acid, tranexamic acid, rutin, and thrombin. Botanical agents having desirable effects on the wound site may also be added.

For use in the present invention, the kaolin (or other clay material) is preferably in powder form and, more preferably, an impalpable (i.e., tactilely undetectable) powder. The present invention is not limited in this regard, however, as other forms of the kaolin such as particles are within the scope of the present invention. As used herein, “particles” include beads, pellets, powders, granules, rods, or any other surface morphology or combination of surface morphologies. Irrespective of the surface morphology, the particles are about 0.0002 mm (millimeters) to about 0.5 mm and preferably about 0.002 mm to about 0.05 mm in effective diameter. The present invention is not limited in this regard, however, and other particle sizes are also within the scope of the present invention.

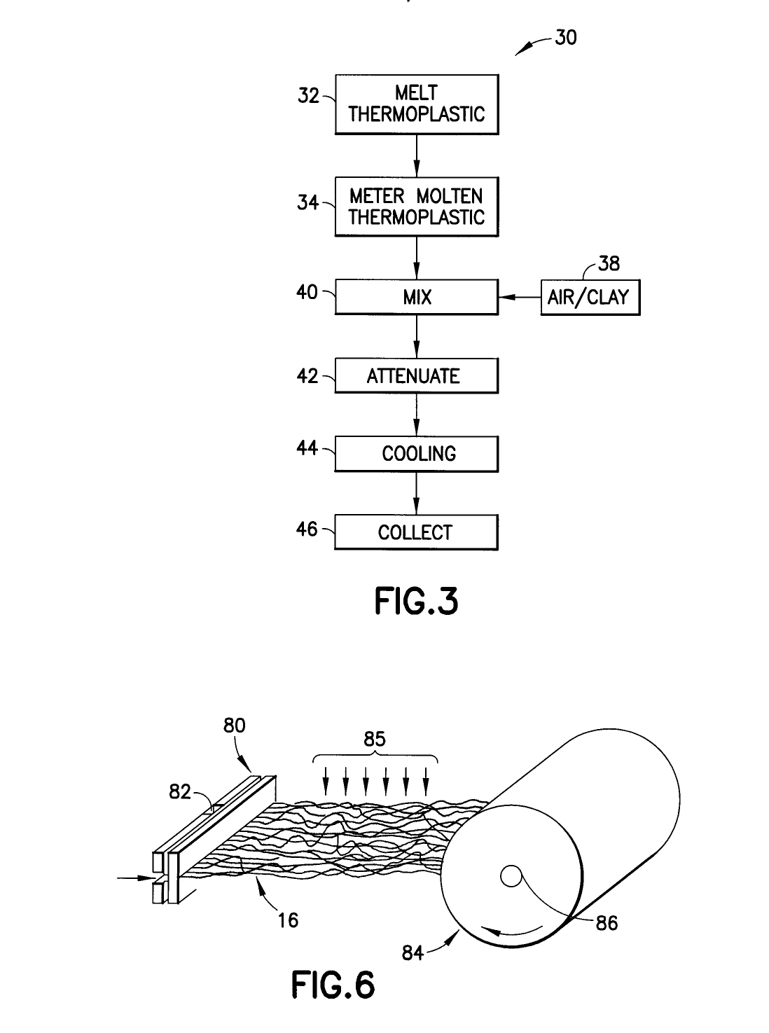

Referring now to FIG. 3, one exemplary process of melt blowing the sponge of the present invention is shown generally at 30 and is hereinafter referred to as “process 30.” Melt blowing is a process in which molten polymer is extruded and drawn with heated, high-velocity air to form fine filaments. The filaments are cooled and collected as a non-woven self-binding web onto a moving screen or conveyor. In the process 30, a polymer is melted (to produce a “melt”) and heated in a melt step 32. In a metering step 34, a predetermined amount of the melt is metered and combined with a hot air stream and clay in a feed step 38 and mixed in a mix step 40. The present invention is not limited to clay as the feed material, however, as other materials as described herein may be used as the hemostatic agent.

From the mix step 40, the combined melt, hot air, and clay is drawn into fine fibers in an attenuation step 42. The fibers are then quenched with surrounding air drawn in a cooling step 44. The fibers are collected in a collection step 46.

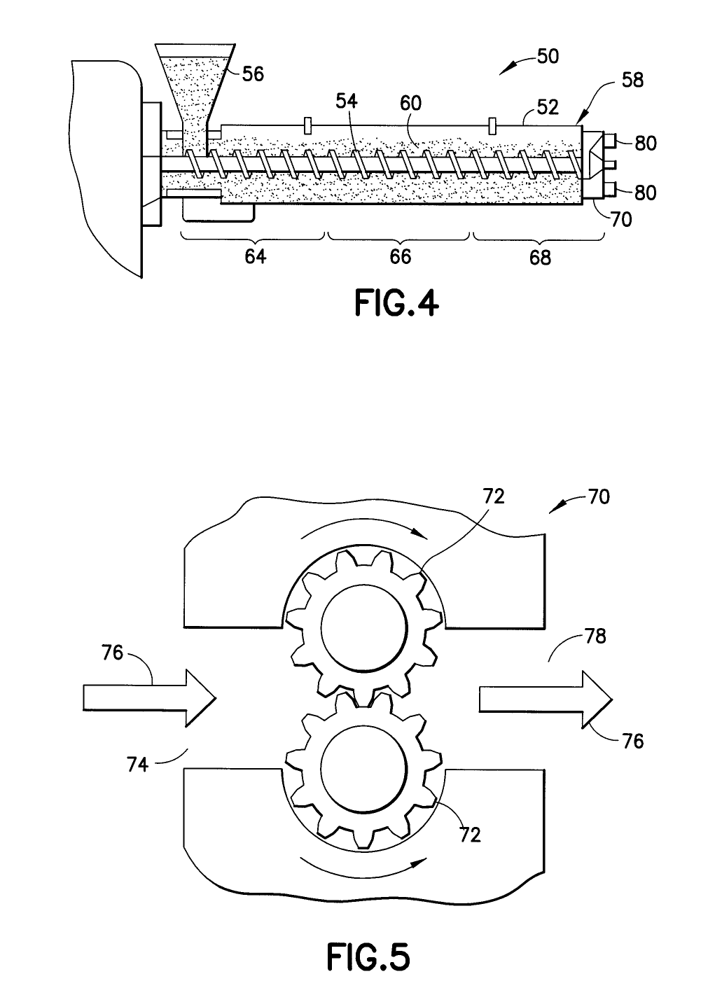

Referring to FIG. 4, the melt step in which the polymer is melted and heated can employ a screw extruder 50. The present invention is not limited in this regard, however, as other devices including other types of extruders can be used. In embodiments utilizing a screw extruder 50, the screw extruder may include a barrel 52 inside which an axially-positioned screw 54 rotates to convey the polymer from a feed hopper 56 to a discharge end, shown generally at 58. A jacket 60 is located over the outside surface of the barrel 52 through which a heated fluid is made to flow to facilitate the melting of the polymer. The present invention is not limited with regard to a jacket through which hot fluid flows, however, as other means of heating the barrel such as heaters, coils of electrically-conductive wire, heat tape, or the like are within the scope of the present invention.

The screw extruder 50 may include three distinct zones, for example, a feed zone 64, a transition zone 66, and a metering zone 68. In the feed zone 64, the polymer is heated and conveyed to the transition zone 66. In the transition zone 66, the distances between the flights of the screw 54 may be decreased so that as the polymer is moved therethrough, the semi-molten and molten polymer is compressed. Any agglomerations of semi-molten polymer is drawn between the outer edges of the screw 54 and the inner surface of the barrel 52 and sheared to homogenize the resulting melt. In the metering zone 68, the polymer is pressurized to allow the melt to attain a suitable pressure for transfer to a die assembly at the discharge end 58. Pressurization of the melt can be effected via a metering pump 70 positioned at the discharge end 58. A die assembly 80 is located downstream of the metering pump 70.

Referring now to FIG. 5, the metering pump 70 can be a positive-displacement and constant-volume device that provides substantially uniform delivery of the melt to the die assembly and ensures consistent flow of the melt under variations in viscosity, pressure, and temperature. The metering pump 70 includes two intermeshing and counter-rotating toothed gears 72, the rotation of which transfers the melt from a suction side 74 (pump inlet at the end of the metering zone) in the direction of arrows 76 to a discharge side 78 (pump outlet at the discharge end of the screw extruder). At the discharge side 78, a uniform flow of the melt is fed to the die assembly.

As shown in FIG. 6, the uniform flow of the melt from the discharge side of the metering pump is received by the die assembly 80. The die assembly 80 also receives the hot air stream, shown at 82. A controlled amount of clay (or other hemostatic agent) may be mixed with the hot air stream 82, or it may be added in a separate stream. As the melt passes through the die assembly 80, the fibers 16 formed thereby are tacky, and the added clay is easily retained thereon. The clay combined with the melt is extruded from the die assembly 80. As the combined clay/polymer is extruded, the hot air stream 82 attenuates the polymer to form the fibers 16. As the fibers 16 progress toward a collector 84, surrounding air 85 is drawn to quench the fibers, and the fibers solidify and are randomly laid on the collector to form the self-bonded non-woven web. Although the fibers 16 are generally laid randomly (and also entangled) because of the turbulence in the hot air stream 82, there is a slight bias in the formation of the web due to the movement of the collector 84. The collector 84 typically comprises a core 86 around which the web is wound.

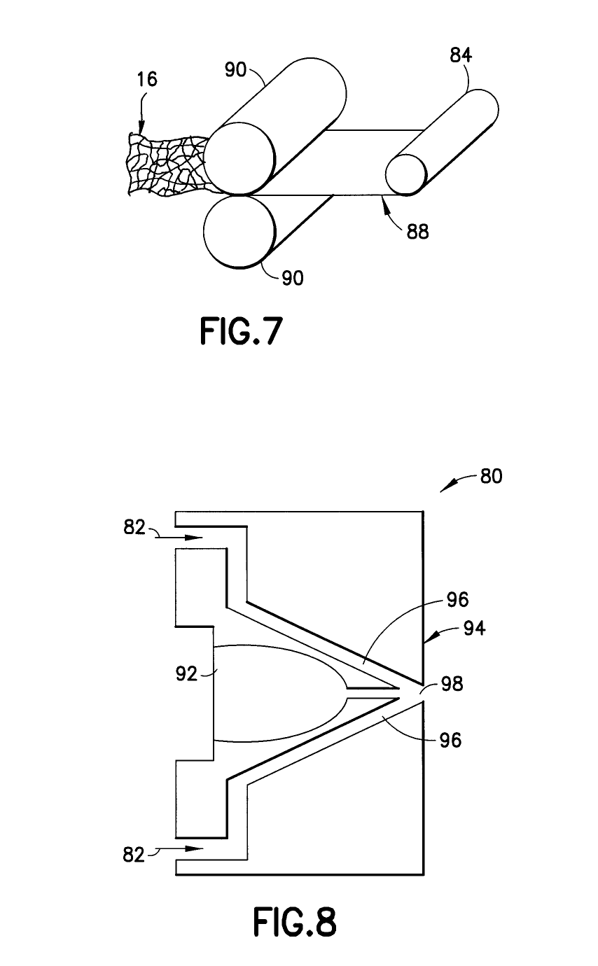

In some embodiments, as is shown in FIG. 7, heated calender and anvil rolls 90 (a series of rolls used to flatten material) can be used to facilitate the formation of the web and to enhance the ability of the fibers 16 to self-bond and to provide for a strong agent/fiber bond without the use of adhesives or binders. As the fibers 16 are laid on the collector 84, they are drawn between the calender and the anvil rolls 90 to intermesh. In some embodiments, the calender and anvil rolls 90 can be patterned to provide a desired surface finish to the web before it is collected on the collector 84.

As shown in FIG. 8, the die assembly 80 can include a distributor 92, a nosepiece 94, and at least one air manifold 96. The distributor 92, which is in fluid communication with the metering pump (if used), is an open channel or trough into which the pressurized melt is received. Because the channel is substantially open at the point at which it is connected to the metering pump, the distributor is known as a “coat hanger-type” of distributor. This type of distributor balances both the flow and residence time across the width of the die assembly 80 and allows a wide variety of materials to be melt blown.

From the distributor 92, the melt is transferred to the nosepiece 94. The nosepiece 94 is a wide, hollow element having orifices 98 extending across the width thereof. The orifices 98 are formed by the junctures of channels extending from the distributor 92 and the air manifolds 96. The particular arrangement of the orifices 98 determines the uniformity of the web formed. The melt is extruded from these orifices 98. In the present invention, the orifices 98 are about 0.05 mm to about 0.8 mm in diameter and spaced at about 1 to about 4 millimeters from each other.

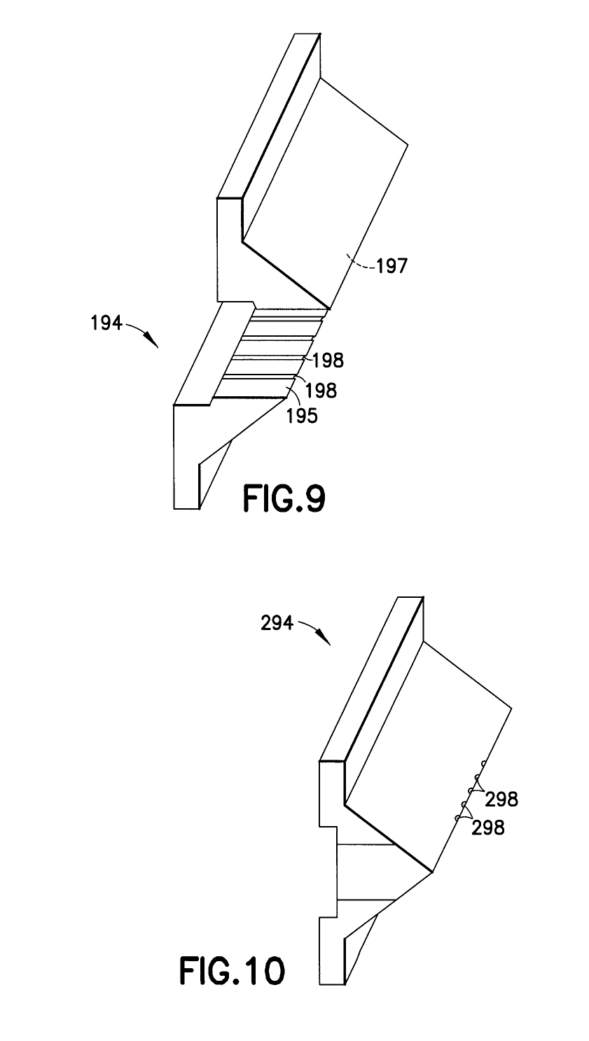

In one embodiment of the nosepiece as shown in FIG. 9, a capillary nosepiece 194 is shown. The capillary nosepiece 194 has a plurality of individual orifices 198 that are actually slots milled into a flat surface 195 and then matched with identical slots milled into a mating surface 197. In another embodiment of the nosepiece as shown in FIG. 10, a drilled hole nosepiece 294 is shown. The drilled hole nosepiece 294 has very small orifices 298 drilled into a block of metal by a mechanical drilling process or by electric discharge machining (EDM).

Referring back to FIG. 8, the air manifold 96 supplies at least one hot air stream 82 through the nosepiece 94 to attenuate the melt to form the fibers. More specifically, the air manifold 96 provides fluid communication between a source of the hot air stream 82 and outlets of the orifices 98. The source of the hot air stream 82 may be an air compressor in which the compressed air is passed through a heat exchanger to heat the air to the desired processing temperature. The temperature of the hot air stream 82 is about 230 degrees C. to about 360 degrees C. (which is typically higher than the melt temperature of the polymer), and the velocity of the hot air stream 82 is about 170 m/s (meters per second) to about 280 m/s.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A device for promoting the clotting of blood, comprising:
a plurality of non-woven fibers of a polymer, said fibers being randomly arranged into a web; and
a hemostatic agent disposed on said fibers;
wherein when treating a bleeding wound, application of said device causes at least a portion of said hemostatic agent to come into contact with blood to cause a clotting effect.

2. The device of claim 1, wherein said non-woven fibers are randomly arranged into said web using a melt blowing technique.

3. The device of claim 1, wherein said hemostatic agent is clay.

4. The device of claim 3, wherein said clay is Edgar's plastic kaolin.

5. The device of claim 1, wherein said hemostatic agent is selected from the group consisting of kaolin, attapulgite, bentonite, zeolite, bioactive glasses, biological hemostats, chitin, chitosan, diatomaceous earth, and combinations of the foregoing.

6. The device of claim 1, further comprising a composition mixed with said hemostatic agent, said composition being selected from the group consisting of magnesium sulfate, sodium metaphosphate, calcium chloride, dextrin, antibiotics, antifungal agents, antimicrobial agents, anti-inflammatory agents, analgesics, antihistamines, compounds containing silver ion, compound containing copper ions, ascorbic acid, tranexamic acid, rutin, thrombin, botanical agents, and combinations of the foregoing.

7. The device of claim 1, wherein said polymer is selected from the group consisting of polypropylenes, polyesters, acrylonitrile butadiene styrene, polyamides, polylactic acid, polyacrylates, and combinations of the foregoing.

8. A hemostatic sponge, comprising:
a melt-blown non-woven fibrous web of a polymer; and
a hemostatic agent attached to fibers of said non-woven fibrous web;
wherein when treating a bleeding wound, application of said sponge causes at least a portion of said hemostatic agent to come into contact with blood to cause a clotting effect.

9. The hemostatic sponge of claim 8, wherein said hemostatic agent is attached to said fibers of said non-woven fibrous web by being added to said polymer in an air stream.

10. The hemostatic sponge of claim 8, wherein said hemostatic agent is clay.

11. The hemostatic sponge of claim 10, wherein said clay is selected from the group consisting of kaolin, attapulgite, bentonite, and combinations of the foregoing.

12. The hemostatic sponge of claim 10, wherein said clay is Edgar's plastic kaolin.

13. The hemostatic sponge of claim 8, wherein said hemostatic agent is selected from the group consisting of clay, bioactive glass, zeolite, biological hemostats, chitin, chitosan, molecular sieve materials, diatomaceous earth, and combinations of the foregoing.

14. The hemostatic sponge of claim 8, wherein said polymer is selected from the group consisting of polypropylenes, polyesters, acrylonitrile butadiene styrene, polyamides, polylactic acid, polyacrylates, and combinations of the foregoing.

15. The hemostatic sponge of claim 8, further comprising a composition mixed with said hemostatic agent, said composition being selected from the group consisting of magnesium sulfate, sodium metaphosphate, calcium chloride, dextrin, antibiotics, antifungal agents, antimicrobial agents, anti-inflammatory agents, analgesics, antihistamines, compounds containing silver ion, compound containing copper ions, ascorbic acid, tranexamic acid, rutin, thrombin, botanical agents, and combinations of the foregoing.

16. The hemostatic sponge of claim 8, wherein said fibers of said non-woven fibrous web are about 0.05 mm to about 0.8 mm in diameter.

17. The hemostatic sponge of claim 8, wherein said fibers of said non-woven fibrous web are about 0.2 mm to about 0.5 mm in diameter.

18. A method of making a hemostatic sponge, said method comprising the steps of:
melting a polymer to produce a melt;
combining said melt with a hot air stream;
adding a hemostatic agent to said melt;
drawing a material formed by a combination of said melt and said hemostatic agent into fibers; and
collecting said fibers as a web.

19. The method of claim 18, further comprising a step of metering said melt through a screw extruder to allow said melt to reach a predetermined pressure before being drawn into fibers.

20. The method of claim 19, wherein said step of metering said melt through said screw extruder comprises pumping said melt from said screw extruder using a metering pump.

21. The method of claim 18, wherein said step of drawing a material formed by a combination of said melt and said hemostatic agent into fibers comprises extruding said material through a die.

22. The method of claim 18, further comprising cooling said material formed by a combination of said melt and said hemostatic agent drawn into fibers prior to collecting said fibers.

23. The method of claim 22, wherein said step of cooling said material comprises drawing surrounding air onto said fibers.

24. The method of claim 18, further comprising heating said melt prior to the step of combining said melt with said hot air stream.

25. The method of claim 18, wherein said step of collecting said fibers as a web comprises winding said web onto a core.

26. The method of claim 18, further comprising flattening said drawn fibers using calender and anvil rolls.

27. The method of claim 26, further comprising heating at least one of said calender and anvil rolls.

28. The method of claim 26, further comprising patterning at least one of said calender and anvil rolls to provide a desired surface finish to said web.
